(19) United States
(12) Patent Application Publication
(10) Pub. No.: US 2026/0197183 A1
(43) Pub. Date: Jul. 9, 2026

(54) ACCESS CONTROL USING TRANSACTIONS

(71) Applicant: nChain Licensing AG, Zug (CH)

(72) Inventors: Craig Steven WRIGHT, London (GB); Bassem AMMAR, London (GB)

(21) Appl. No.: 19/132,749

(22) PCT Filed: Nov. 15, 2023
(86) PCT No.: PCT/EP2023/081939

§ 371 Date: May 23, 2025

(30) Foreign Application Priority Data
Nov. 25, 2022 (GB) 2217665.5

(51) Int. Cl. H04L 9/32; G16H 10/60; H04L 9/00; H04L 9/08

(52) U.S. Cl. CPC H04L 9/3247; G16H 10/60; H04L 9/083; H04L 9/0841; H04L 9/50

(57) ABSTRACT

A method performed by a first party, the method comprising: receiving, from a second party, information of a first blockchain transaction, the first blockchain transaction comprising an input and an output, wherein the input of the first blockchain transaction comprises a signature of the second party and wherein the output of the first blockchain transaction is locked to a public key of the first party; performing an action comprising at least one of: receiving authorisation of access to a product or service; issuing authorisation of access to a product or service; providing a product or service; receiving a product or service; in response to performing the action, providing, as part of a first input of a second blockchain transaction, a signature of the first party to unlock the output of the first blockchain transaction

### CROSS REFERENCE TO RELATED APPLICATIONS

This application is the U.S. National Stage of International Application No. PCT/EP2023/081939 filed on Nov. 15, 2023, which claims the benefit of United Kingdom Patent Application No. 2217665.5, filed on Nov. 25, 2022, the contents of which are all incorporated herein by reference in their entireties.

### TECHNICAL FIELD

The present disclosure relates to databases and the use of databases.

### BACKGROUND

A blockchain refers to a form of distributed data structure, wherein a duplicate copy of the blockchain is maintained at each of a plurality of nodes in a distributed peer-to-peer (P2P) network (referred to below as a “blockchain network”) and widely publicised. The blockchain comprises a chain of blocks of data, wherein each block comprises one or more transactions. Each transaction, other than so-called “coinbase transactions”, points back to a preceding transaction in a sequence which may span one or more blocks going back to one or more coinbase transactions. Coinbase transactions are discussed further below. Transactions that are submitted to the blockchain network are included in new blocks. New blocks are created by a process often referred to as “mining”, which involves each of a plurality of the nodes competing to perform “proof-of-work”, i.e. solving a cryptographic puzzle based on a representation of a defined set of ordered and validated pending transactions waiting to be included in a new block of the blockchain. It should be noted that the blockchain may be pruned at some nodes, and the publication of blocks can be achieved through the publication of mere block headers.

The transactions in the blockchain may be used for one or more of the following purposes: to convey a digital asset (i.e. a number of digital tokens), to order a set of entries in a virtualised ledger or registry, to receive and process timestamp entries, and/or to time-order index pointers. A blockchain can also be exploited in order to layer additional functionality on top of the blockchain. For example, blockchain protocols may allow for storage of additional user data or indexes to data in a transaction. There is no pre-specified limit to the maximum data capacity that can be stored within a single transaction, and therefore increasingly more complex data can be incorporated. For instance this may be used to store an electronic document in the blockchain, or audio or video data.

In an “output-based” model (sometimes referred to as a UTXO-based model), the data structure of a given transaction comprises one or more inputs and one or more outputs. Any spendable output comprises an element specifying an amount of the digital asset that is derivable from the proceeding sequence of transactions. The spendable output is sometimes referred to as a UTXO (“unspent transaction output”). The output may further comprise a locking script specifying a condition for the future redemption of the output. A locking script is a predicate defining the conditions necessary to validate and transfer digital tokens or assets. Each input of a transaction (other than a coinbase transaction) comprises a pointer (i.e. a reference) to such an output in a preceding transaction, and may further comprise an unlocking script for unlocking the locking script of the pointed-to output. So consider a pair of transactions, call them a first and a second transaction (or “target” transaction). The first transaction comprises at least one output specifying an amount of the digital asset, and comprising a locking script defining one or more conditions of unlocking the output. The second, target transaction comprises at least one input, comprising a pointer to the output of the first transaction, and an unlocking script for unlocking the output of the first transaction.

In such a model, when the second, target transaction is sent to the blockchain network to be propagated and recorded in the blockchain, one of the criteria for validity applied at each node will be that the unlocking script meets all of the one or more conditions defined in the locking script of the first transaction. Another will be that the output of the first transaction has not already been redeemed by another, earlier valid transaction. Any node that finds the target transaction invalid according to any of these conditions will not propagate it (as a valid transaction, but possibly to register an invalid transaction) nor include it in a new block to be recorded in the blockchain.

An alternative type of transaction model is an account-based model. In this case each transaction does not define the amount to be transferred by referring back to the UTXO of a preceding transaction in a sequence of past transactions, but rather by reference to an absolute account balance. The current state of all accounts is stored by the nodes separate to the blockchain and is updated constantly.

### Translucent Databases

Translucent database is a term used in “Peter Wayner, Translucent Databases 2^{nd} Edition: Confusion, Misdirection, Randomness, Sharing, Authentication And Steganography To Defend Privacy—Jan. 8, 2009” to describe techniques to design privacy-preserving database at low complexity cost. The Linux password file is often given as a practical example of translucent database. Salted hash of the user's password is saved rather than the password in the cleartext. To grant access, the user enters his password to the system, which hashes and compares it against the one it stores. Only the user knows the password. The system keeps the salted hash and does not keep the pre-image (i.e. the cleartext password). Anyone other than the password owner would have to guess the pre-image. The system is more secure against insider's attack, since anyone who can access the password file will find only hashes. Thus the main idea is to store hash (password) and use it in granting access rather than the password itself.

### Merkle Proof

It has been shown in that the Merkle proof of a transaction proves the existence of said transaction on the blockchain as well as the existence of the transactions whose outpoints are spent in said transaction. FIG. 3 shows an example transaction graph. As illustrated in FIG. 3, if transaction txᵢ has two inputs outpointⱼ=txIdⱼ|index and outpointₖ=txIdₖ|index then the Merkle proof of txᵢ proofs their existence (i.e. that txᵢ is a confirmed transaction in the blockchain). Also if txᵢ existence is proven and its raw transaction form is provided, this also proves the existence of txⱼ and txₖ. We call transaction txt a child transaction, and we call transactions txⱼ, txₖ parent transactions. Furthermore if txⱼ in its raw transaction form is available, this also proves the existence of all parent transactions of txⱼ, and so on.

In FIG. 3 Error! Reference source not found., a transaction is represented a circle. An arrow is shown from a parent transaction to a child transaction (e.g. txᵢ is a child of txⱼ and txₖ). To prove the existence (on the blockchain) of txₘ, txₗ, txⱼ, txₖ, we do not need to provide the Merkle proof of each transaction. Instead, we only need to provide the Merkle proof of txᵢ and the raw transactions forms of txᵢ, txⱼ and txₗ. Note that we do not need the raw transaction form of txₖ and txₘ, we only need their txID (the hash of the transaction). Note also that proving the existence of txₗ does not prove the existence of its child txₙ.

### 3-Party Key Generation

When two parties Alice and Bob want to communicate confidentially, they can use the Diffie Hellman key exchange protocol to generate a secret key K_{symetric} and use it in symmetric key encryption. In this setting Alice and Bob have public, private key pairs (s_{A}, K_{A}=s_{A}G), and (s_{B}, K_{B}=s_{B}G), respectively, where K_{A}, K_{B}, G are elliptic curve points.

Alice and Bob can generate asymmetric key in one round of communication.
1. Alice sends Bob her public key K_{A},
2. Bob sends Alice his public key K_{B}
   Alice calculates the symmetric key using K_{symmetric}=s_{A}K_{B}
   Bob calculates the symmetric key using K_{symmetric}=s_{B}K_{A}

The symmetric key is given by K_{symmetric}=s_{A}K_{B}=s_{B}K_{A}=s_{A}s_{B}G. Note that Alice and Bob did not share their private keys at any point. Note also that we assume that the integrity of the channel is maintained i.e. Alice and Bob ensure they are getting each other public key.

To allow 3-party key generation between Alice, Bob and Charly, we can optionally use three-party key agreement protocol DHP. For example the symmetric key can be given by

$${K}_{symmetric} = {s}_{A} {s}_{B} {s}_{C} G = {s}_{B} {s}_{C} {K}_{A} = {s}_{A} {s}_{B} {K}_{C} = {s}_{A} {s}_{C} {K}_{B}$$

This can be calculated in two rounds.
1. In the first round
   a. Alice sends K_{A} to Bob
   b. Bob sends K_{B} to Charly
   c. Charly sends K_{C} to Alice
2. In the second round
   a. Alice sends s_{A}K_{C} to Bob, who can then calculate K_{symmetric}=s_{B}s_{A}K_{C}
   b. Bob sends s_{B} K_{A} to Charly, who can then calculate K_{symmetric}=s_{C}s_{B}K_{A}
   c. Charly sends s_{C}K_{B} to Alice, who can then calculate K_{symmetric}=s_{A}s_{C}K_{B}

At the end of the second round each party can calculate the symmetric key. Also note that no private keys are shared.

It is possible to use bilinear pairing such that the encryption key is given by K_{symmetric}=ê(K_{A}, K_{B})^{s}^{_{C}}=ê(K_{A}, K_{C}) s_{B}=ê(K_{B}, K_{C}) s_{A}.

Here each of the three parties needs only its own secret key and the public keys of the other two parties to calculate K_{symmetric}. It should be noted that bilinear mapping is not suitable with the Secp256k1 parameters.

### SUMMARY

According to one aspect disclosed herein, there is provided a method performed by a first party. The method comprises receiving, from a second party, information of a first blockchain transaction, the first blockchain transaction comprising an input and an output, wherein the input of the first blockchain transaction comprises a signature of the second party and wherein the output of the first blockchain transaction is locked to a public key of the first party. The method may also include performing an action comprising at least one of: receiving authorisation of access to a product or service; issuing authorisation of access to a product or service; providing a product or service; receiving a product or service. The method may also comprise, in response to performing the action, providing, as part of a first input of a second blockchain transaction, a signature of the first party to unlock the output of the first blockchain transaction.

### BRIEF DESCRIPTION OF THE DRAWINGS

To assist understanding of embodiments of the present disclosure and to show how such embodiments may be put into effect, reference is made, by way of example only, to the accompanying drawings in which:

FIG. 1 is a schematic block diagram of a system for implementing a blockchain,

FIG. 2 schematically illustrates some examples of transactions which may be recorded in a blockchain,

FIG. 3 shows an example transaction graph;

FIG. 4 shows an example transaction graph;

FIG. 5 shows an example transaction graph;

FIG. 6 shows an example stream of events;

FIG. 7 shows an example stream of events;

FIG. 8 shows an example method for creating a child-of-all transaction;

FIG. 9 shows an example method.

### DETAILED DESCRIPTION OF EMBODIMENTS

### 1. Example System Overview

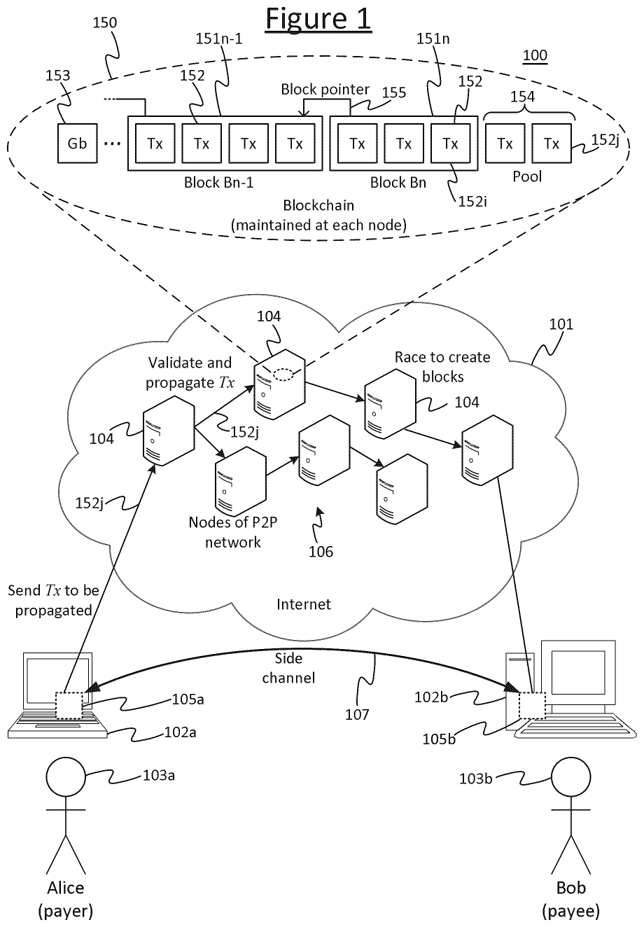

FIG. 1 shows an example system 100 for implementing a blockchain 150. The system 100 may comprise a packet-switched network 101, typically a wide-area internetwork such as the Internet. The packet-switched network 101 comprises a plurality of blockchain nodes 104 that may be arranged to form a peer-to-peer (P2P) network 106 within the packet-switched network 101. Whilst not illustrated, the blockchain nodes 104 may be arranged as a near-complete graph. Each blockchain node 104 is therefore highly connected to other blockchain nodes 104.

Each blockchain node 104 comprises computer equipment of a peer, with different ones of the nodes 104 belonging to different peers. Each blockchain node 104 comprises processing apparatus comprising one or more processors, e.g. one or more central processing units (CPUs), accelerator processors, application specific processors and/or field programmable gate arrays (FPGAs), and other equipment such as application specific integrated circuits (ASICs). Each node also comprises memory, i.e. computer-readable storage in the form of a non-transitory computer-readable medium or media. The memory may comprise one or more memory units employing one or more memory media, e.g. a magnetic medium such as a hard disk; an electronic medium such as a solid-state drive (SSD), flash memory or EEPROM; and/or an optical medium such as an optical disk drive.

The blockchain 150 comprises a chain of blocks of data 151, wherein a respective copy of the blockchain 150 is maintained at each of a plurality of blockchain nodes 104 in the distributed or blockchain network 106. As mentioned above, maintaining a copy of the blockchain 150 does not necessarily mean storing the blockchain 150 in full. Instead, the blockchain 150 may be pruned of data so long as each blockchain node 150 stores the block header (discussed below) of each block 151. Each block 151 in the chain comprises one or more transactions 152, wherein a transaction in this context refers to a kind of data structure. The nature of the data structure will depend on the type of transaction protocol used as part of a transaction model or scheme. A given blockchain will use one particular transaction protocol throughout. In one common type of transaction protocol, the data structure of each transaction 152 comprises at least one input and at least one output. Each output specifies an amount representing a quantity of a digital asset as property, an example of which is a user 103 to whom the output is cryptographically locked (requiring a signature or other solution of that user in order to be unlocked and thereby redeemed or spent). Each input points back to the output of a preceding transaction 152, thereby linking the transactions.

Each block 151 also comprises a block pointer 155 pointing back to the previously created block 151 in the chain so as to define a sequential order to the blocks 151. Each transaction 152 (other than a coinbase transaction) comprises a pointer back to a previous transaction so as to define an order to sequences of transactions (N.B. sequences of transactions 152 are allowed to branch). The chain of blocks 151 goes all the way back to a genesis block (Gb) 153 which was the first block in the chain. One or more original transactions 152 early on in the chain 150 pointed to the genesis block 153 rather than a preceding transaction.

A blockchain node 104 may be configured to forward transactions 152 to other blockchain nodes 104, and thereby cause transactions 152 to be propagated throughout the network 106. A blockchain node 104 may be configured to create blocks 151 and to store a respective copy of the same blockchain 150 in their respective memory. A blockchain node 104 may also maintain an ordered set (or “pool”) 154 of transactions 152 waiting to be incorporated into blocks 151. The ordered pool 154 is often referred to as a “mempool”. This term herein is not intended to limit to any particular blockchain, protocol or model. It refers to the ordered set of transactions which a node 104 has accepted as valid and for which the node 104 is obliged not to accept any other transactions attempting to spend the same output.

In a given present transaction 152j, the (or each) input comprises a pointer referencing the output of a preceding transaction 152i in the sequence of transactions, specifying that this output is to be redeemed or “spent” in the present transaction 152j. Spending or redeeming does not necessarily imply transfer of a financial asset, though that is certainly one common application. More generally spending could be described as consuming the output, or assigning it to one or more outputs in another, onward transaction. In general, the preceding transaction could be any transaction in the ordered set 154 or any block 151. The preceding transaction 152i need not necessarily exist at the time the present transaction 152j is created or even sent to the network 106, though the preceding transaction 152i will need to exist and be validated in order for the present transaction to be valid. Hence “preceding” herein refers to a predecessor in a logical sequence linked by pointers, not necessarily the time of creation or sending in a temporal sequence, and hence it does not necessarily exclude that the transactions 152i, 152j be created or sent out-of-order (see discussion below on orphan transactions). The preceding transaction 152i could equally be called the antecedent or predecessor transaction.

The input of the present transaction 152j also comprises the input authorisation, for example the signature of the user 103a to whom the output of the preceding transaction 152i is locked. In turn, the output of the present transaction 152j can be cryptographically locked to a new user or entity 103b. The present transaction 152j can thus transfer the amount defined in the input of the preceding transaction 152i to the new user or entity 103b as defined in the output of the present transaction 152j. In some cases a transaction 152 may have multiple outputs to split the input amount between multiple users or entities (one of whom could be the original user or entity 103a in order to give change). In some cases a transaction can also have multiple inputs to gather together the amounts from multiple outputs of one or more preceding transactions, and redistribute to one or more outputs of the current transaction.

Due to the resources involved in transaction validation and publication, typically at least each of the blockchain nodes 104 takes the form of a server comprising one or more physical server units, or even whole a data centre. However in principle any given blockchain node 104 could take the form of a user terminal or a group of user terminals networked together.

The memory of each blockchain node 104 stores software configured to run on the processing apparatus of the blockchain node 104 in order to perform its respective role or roles and handle transactions 152 in accordance with the blockchain node protocol. It will be understood that any action attributed herein to a blockchain node 104 may be performed by the software run on the processing apparatus of the respective computer equipment. The node software may be implemented in one or more applications at the application layer, or a lower layer such as the operating system layer or a protocol layer, or any combination of these.

Also connected to the network 101 is the computer equipment 102 of each of a plurality of parties 103 in the role of consuming users. These users may interact with the blockchain network 106 but do not participate in validating transactions or constructing blocks. Some of these users or agents 103 may act as senders and recipients in transactions. Other users may interact with the blockchain 150 without necessarily acting as senders or recipients. For instance, some parties may act as storage entities that store a copy of the blockchain 150 (e.g. having obtained a copy of the blockchain from a blockchain node 104).

Some or all of the parties 103 may be connected as part of a different network, e.g. a network overlaid on top of the blockchain network 106. Users of the blockchain network (often referred to as “clients”) may be said to be part of a system that includes the blockchain network 106; however, these users are not blockchain nodes 104 as they do not perform the roles required of the blockchain nodes. Instead, each party 103 may interact with the blockchain network 106 and thereby utilize the blockchain 150 by connecting to (i.e. communicating with) a blockchain node 106. Two parties 103 and their respective equipment 102 are shown for illustrative purposes: a first party 103a and his/her respective computer equipment 102a, and a second party 103b and his/her respective computer equipment 102b. It will be understood that many more such parties 103 and their respective computer equipment 102 may be present and participating in the system 100, but for convenience they are not illustrated. Each party 103 may be an individual or an organization. Purely by way of illustration the first party 103a is referred to herein as Alice and the second party 103b is referred to as Bob, but it will be appreciated that this is not limiting and any reference herein to Alice or Bob may be replaced with “first party” and “second “party” respectively.

The computer equipment 102 of each party 103 comprises respective processing apparatus comprising one or more processors, e.g. one or more CPUs, GPUs, other accelerator processors, application specific processors, and/or FPGAs. The computer equipment 102 of each party 103 further comprises memory, i.e. computer-readable storage in the form of a non-transitory computer-readable medium or media. This memory may comprise one or more memory units employing one or more memory media, e.g. a magnetic medium such as hard disk; an electronic medium such as an SSD, flash memory or EEPROM; and/or an optical medium such as an optical disc drive. The memory on the computer equipment 102 of each party 103 stores software comprising a respective instance of at least one client application 105 arranged to run on the processing apparatus. It will be understood that any action attributed herein to a given party 103 may be performed using the software run on the processing apparatus of the respective computer equipment 102. The computer equipment 102 of each party 103 comprises at least one user terminal, e.g. a desktop or laptop computer, a tablet, a smartphone, or a wearable device such as a smartwatch. The computer equipment 102 of a given party 103 may also comprise one or more other networked resources, such as cloud computing resources accessed via the user terminal.

The client application 105 may be initially provided to the computer equipment 102 of any given party 103 on suitable computer-readable storage medium or media, e.g. downloaded from a server, or provided on a removable storage device such as a removable SSD, flash memory key, removable EEPROM, removable magnetic disk drive, magnetic floppy disk or tape, optical disk such as a CD or DVD ROM, or a removable optical drive, etc.

The client application 105 comprises at least a “wallet” function. This has two main functionalities. One of these is to enable the respective party 103 to create, authorise (for example sign) and send transactions 152 to one or more bitcoin nodes 104 to then be propagated throughout the network of blockchain nodes 104 and thereby included in the blockchain 150. The other is to report back to the respective party the amount of the digital asset that he or she currently owns. In an output-based system, this second functionality comprises collating the amounts defined in the outputs of the various 152 transactions scattered throughout the blockchain 150 that belong to the party in question.

Note: whilst the various client functionality may be described as being integrated into a given client application 105, this is not necessarily limiting and instead any client functionality described herein may instead be implemented in a suite of two or more distinct applications, e.g. interfacing via an API, or one being a plug-in to the other. More generally the client functionality could be implemented at the application layer or a lower layer such as the operating system, or any combination of these. The following will be described in terms of a client application 105 but it will be appreciated that this is not limiting.

The instance of the client application or software 105 on each computer equipment 102 is operatively coupled to at least one of the blockchain nodes 104 of the network 106. This enables the wallet function of the client 105 to send transactions 152 to the network 106. The client 105 is also able to contact blockchain nodes 104 in order to query the blockchain 150 for any transactions of which the respective party 103 is the recipient (or indeed inspect other parties' transactions in the blockchain 150, since in embodiments the blockchain 150 is a public facility which provides trust in transactions in part through its public visibility). The wallet function on each computer equipment 102 is configured to formulate and send transactions 152 according to a transaction protocol. As set out above, each blockchain node 104 runs software configured to validate transactions 152 according to the blockchain node protocol, and to forward transactions 152 in order to propagate them throughout the blockchain network 106. The transaction protocol and the node protocol correspond to one another, and a given transaction protocol goes with a given node protocol, together implementing a given transaction model. The same transaction protocol is used for all transactions 152 in the blockchain 150. The same node protocol is used by all the nodes 104 in the network 106.

An alternative type of transaction protocol operated by some blockchain networks may be referred to as an “account-based” protocol, as part of an account-based transaction model. In the account-based case, each transaction does not define the amount to be transferred by referring back to the UTXO of a preceding transaction in a sequence of past transactions, but rather by reference to an absolute account balance. The current state of all accounts is stored, by the nodes of that network, separate to the blockchain and is updated constantly. In such a system, transactions are ordered using a running transaction tally of the account (also called the “position”). This value is signed by the sender as part of their cryptographic signature and is hashed as part of the transaction reference calculation. In addition, an optional data field may also be signed the transaction. This data field may point back to a previous transaction, for example if the previous transaction ID is included in the data field.

### 2. UTXO-Based Model

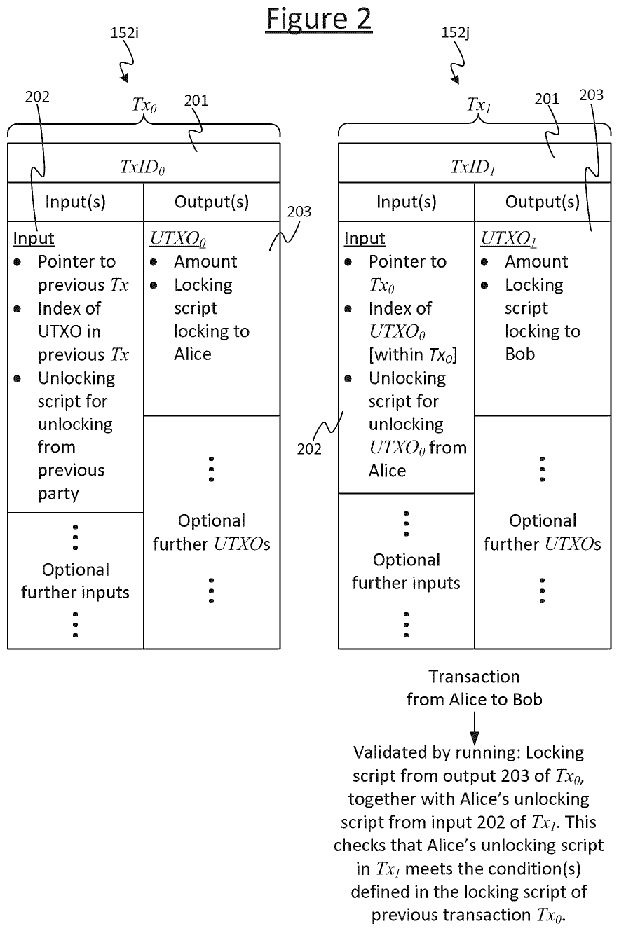

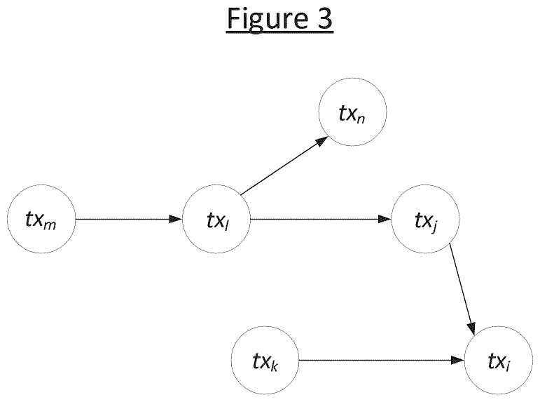

FIG. 2 illustrates an example transaction protocol. This is an example of a UTXO-based protocol. A transaction 152 (abbreviated “Tx”) is the fundamental data structure of the blockchain 150 (each block 151 comprising one or more transactions 152). The following will be described by reference to an output-based or “UTXO” based protocol. However, this is not limiting to all possible embodiments. Note that while the example UTXO-based protocol is described with reference to bitcoin, it may equally be implemented on other example blockchain networks.

In a UTXO-based model, each transaction (“Tx”) 152 comprises a data structure comprising one or more inputs 202, and one or more outputs 203. Each output 203 may comprise an unspent transaction output (UTXO), which can be used as the source for the input 202 of another new transaction (if the UTXO has not already been redeemed). The UTXO includes a value specifying an amount of a digital asset. This represents a set number of tokens on the distributed ledger. The UTXO may also contain the transaction ID of the transaction from which it came, amongst other information. The transaction data structure may also comprise a header 201, which may comprise an indicator of the size of the input field(s) 202 and output field(s) 203. The header 201 may also include an ID of the transaction. In embodiments the transaction ID is the hash of the transaction data (excluding the transaction ID itself) and stored in the header 201 of the raw transaction 152 submitted to the nodes 104.

Say Alice 103a wishes to create a transaction 152j transferring an amount of the digital asset in question to Bob 103b. In FIG. 2 Alice's new transaction 152j is labelled “Tx₁”. It takes an amount of the digital asset that is locked to Alice in the output 203 of a preceding transaction 152i in the sequence, and transfers at least some of this to Bob. The preceding transaction 152i is labelled “Tx₀” in FIG. 2. Tx₀ and Tx₁ are just arbitrary labels. They do not necessarily mean that Tx₀ is the first transaction in the blockchain 151, nor that Tx₁ is the immediate next transaction in the pool 154. Tx₁ could point back to any preceding (i.e. antecedent) transaction that still has an unspent output 203 locked to Alice.

The terms “preceding” and “subsequent” as used herein in the context of the sequence of transactions refer to the order of the transactions in the sequence as defined by the transaction pointers specified in the transactions (which transaction points back to which other transaction, and so forth). They could equally be replaced with “predecessor” and “successor”, or “antecedent” and “descendant”, “parent” and “child”, or such like. It does not necessarily imply an order in which they are created, sent to the network 106, or arrive at any given blockchain node 104. Nevertheless, a subsequent transaction (the descendent transaction or “child”) which points to a preceding transaction (the antecedent transaction or “parent”) will not be validated until and unless the parent transaction is validated. A child that arrives at a blockchain node 104 before its parent is considered an orphan. It may be discarded or buffered for a certain time to wait for the parent, depending on the node protocol and/or node behaviour.

One of the one or more outputs 203 of the preceding transaction Tx₀ comprises a particular UTXO, labelled here UTXO₀. Each UTXO comprises a value specifying an amount of the digital asset represented by the UTXO, and a locking script which defines a condition which must be met by an unlocking script in the input 202 of a subsequent transaction in order for the subsequent transaction to be validated, and therefore for the UTXO to be successfully redeemed.

The locking script (aka scriptPubKey) is a piece of code written in the domain specific language recognized by the node protocol. A particular example of such a language is called “Script” (capital S) which is used by the blockchain network. The locking script specifies what information is required to spend a transaction output 203, for example the requirement of Alice's signature. Locking scripts appear in the outputs of transactions. The unlocking script (aka scriptSig) is a piece of code written the domain specific language that provides the information required to satisfy the locking script criteria. For example, it may contain Bob's signature. Unlocking scripts appear in the input 202 of transactions.

So in the example illustrated, UTXO₀ in the output 203 of Tx₀ comprises a locking script [Checksig P_{A}] which requires a signature Sig P_{A} of Alice in order for UTXO₀ to be redeemed (strictly, in order for a subsequent transaction attempting to redeem UTXO₀ to be valid). [Checksig P_{A}] contains a representation (i.e. a hash) of the public key P_{A} from a public-private key pair of Alice. The input 202 of Tx₁ comprises a pointer pointing back to Tx₁ (e.g. by means of its transaction ID, TxID₀, which in embodiments is the hash of the whole transaction Tx₀). The input 202 of Tx₁ comprises an index identifying UTXO₀ within Tx₀, to identify it amongst any other possible outputs of Tx₀. The input 202 of Tx₁ further comprises an unlocking script <Sig P_{A}> which comprises a cryptographic signature of Alice, created by Alice applying her private key from the key pair to a predefined portion of data (sometimes called the “message” in cryptography). The data (or “message”) that needs to be signed by Alice to provide a valid signature may be defined by the locking script, or by the node protocol, or by a combination of these.

When the new transaction Tx₁ arrives at a blockchain node 104, the node applies the node protocol. This comprises running the locking script and unlocking script together to check whether the unlocking script meets the condition defined in the locking script (where this condition may comprise one or more criteria).

Note that the script code is often represented schematically (i.e. not using the exact language). For example, one may use operation codes (opcodes) to represent a particular function. “OP_. . . ” refers to a particular opcode of the Script language. As an example, OP_RETURN is an opcode of the Script language that when preceded by OP_FALSE at the beginning of a locking script creates an unspendable output of a transaction that can store data within the transaction, and thereby record the data immutably in the blockchain 150. E.g. the data could comprise a document which it is desired to store in the blockchain.

Typically an input of a transaction contains a digital signature corresponding to a public key P_{A}. In embodiments this is based on the ECDSA using the elliptic curve secp256k1. A digital signature signs a particular piece of data. In some embodiments, for a given transaction the signature will sign part of the transaction input, and some or all of the transaction outputs. The particular parts of the outputs it signs depends on the SIGHASH flag. The SIGHASH flag is usually a 4-byte code included at the end of a signature to select which outputs are signed (and thus fixed at the time of signing).

The locking script is sometimes called “scriptPubKey” referring to the fact that it typically comprises the public key of the party to whom the respective transaction is locked. The unlocking script is sometimes called “scriptSig” referring to the fact that it typically supplies the corresponding signature. However, more generally it is not essential in all applications of a blockchain 150 that the condition for a UTXO to be redeemed comprises authenticating a signature. More generally the scripting language could be used to define any one or more conditions. Hence the more general terms “locking script” and “unlocking script” may be preferred.

### 3. Blockchain Database

Examples describe creating a database securely on a blockchain. In some examples, the database comprise a translucent blockchain database. Some examples create a secret and distributed system that is used to access and allocate product/services while also maintaining privacy for the participants.

Some examples relate a medical/drug database used to access and allocate medicine.

According to a first example (Section 3.1), intersecting event streams (sequence of events) are provided. Each actor (e.g., a doctor, a pharmacist and a patient) has their own event stream for their actions, where each event can be recorded in a blockchain transaction. When an interaction happens between two or more actors, it is mapped as an intersection of their event streams. Each actor's activity can be traced by following the transaction graph.

According to a second example (Section 3.2), a mapping between a sequence of events and blockchain transactions is hidden from a public observer. The mapping can be hidden off-chain only, or on-chain as well. One way to have the on-chain mapping is to publish with the current event, e_{A}_{ᵢ}, the hash of an unspent outpoint Hash(outpointₐ), in the event-publishing-transaction (EPT)

$${tx}_{{e}_{{A}_{i + 1}}}$$

The outpoint outpointₐ can then be used as an input to the EPT,

$${tx}_{{e}_{{A}_{i + 1}}} ,$$

that is publishing the next event in the stream A. i.e. The information about the EPT,

$${tx}_{{e}_{{A}_{i + 1}}}$$

publishing the next event, e_{A}_{ᵢ₊₁}, is hidden in the EPT,

$${tx}_{{e}_{{A}_{i}}} ,$$

publishing the current event. Note that

$${tx}_{{e}_{{A}_{i}}} .$$

is not spending an output of

$${tx}_{{e}_{{A}_{i}}} .$$

i.e. they do not have a child-parent relationship.

The second example improves privacy however it does not allow one to efficiently prove events occurred. When EPTs have child-parent relationship, proving the existence of the child transaction (by providing its Merkle proof) proves the existence of the parent transaction (without need to provide the Merkle proof of the parent transaction). When EPTs do not have a child-parent relationship, this method cannot be used to prove existence of the parent transaction, so in one solution Merkle proof of each EPT can be provided to prove existence. In some examples, a child-of-all transaction tx_{coa} is created that spends an outpoint from each EPT. The Merkle proof of tx_{coa} can be used to prove the existence of all EPTs. In some examples reduction of size of tx_{coa} is also provided. In some examples, to minimize privacy risks, we combine EPTs of different streams in one single tx_{coa}.

In a third example (Section 3.4), unspent transaction outputs are used as a token to control access to a database. An unspent output indicates a valid token, and a spent one indicates invalidity. In an example, we show how a patient (data record owner) using these tokens can permit access to doctors and pharmacists to view and update the patient's database records. We also show that these tokens can be used to hide signer's identity.

### 3.1 Intersecting Event Streams (“First Layer Solution”)

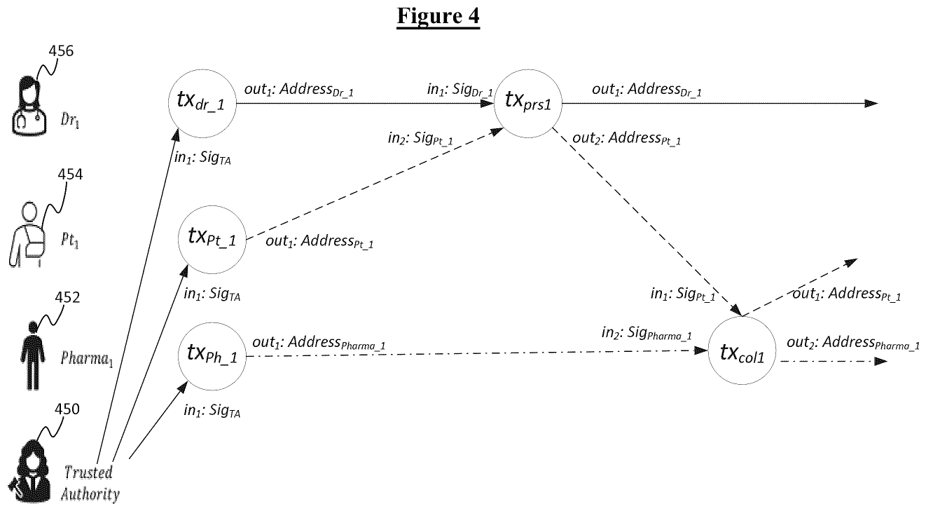

FIG. 4 shows an example graph. A circle represents a transaction, and arrows pointing in and out of a circle represent inputs and outputs respectively.

In FIG. 4, four parties 450, 452, 454 and 456 are provided. Signatures can be used to authenticate events. Signatures can be included in the unlocking script of transactions. Each entity 450, 452, 454 and 456 may have their own blockchain wallet. Each entity 450, 452, 454 and 456 may be able to prove its identity using a registered key pair linked to the identity and role of the respective entity. Party 450 may comprise a trusted authority (e.g., Kensei). A trusted authority may comprise a trusted identification and authentication authority. According to some examples, party 452 may comprise a provider of a product and/or a service. According to some examples, party 454 may comprise a user of the product and/or a service. According to some examples, party 454 may comprise an authoriser of access to the provider and/or a service.

In the specific example shown in FIG. 4, party 450 comprises a trusted authority, party 452 comprises a pharmacy, party 454 comprises a patient and party 456 comprises a doctor. It should be noted however that these are examples only, and in other examples, each party may comprise a different kind of entity.

Each transaction may publish an event. Events may include, for example:
1. Registration: each actor's public key is paired to its identity.
2. Authorisation: An authoriser views a consumer history and issues authorisation for a product or service.
3. Collection: A provider checks a consumer history, checks the authorisation is signed by an authorized actor and gives the product/service to the consumer.
4. Auditing: An auditor checks that each actor is carrying its duties diligently.

In a medical example, the event may include, for example:
1. Registration: each actor's public key is paired to its identity.
2. Prescription issuing: The doctor views the patient history and issues a prescription.
3. Collection: The pharmacist views the patient's history, checks the prescription is signed by an authorized actor and give the prescription to the patient.
4. Auditing: An auditor checks that each actor is carrying its duties diligently.

FIG. 4 shows three registration EPTs (identification and authentication EPTs). Registration events are published in transactions tx_{Dr}_{₁}, tx_{Pt}_{₁}, txₚₕ_{₁}. Each transaction is signed by trusted authority 450 and contains relevant information about the identified subject and role. The output of each transaction can only be spent by the identified subject. The input of each identification and authentication event transactions may comprise a signature of trusted authority 450. The output of the transaction may be locked to a public key of the party identified in the registration event.

For example: tx_{Dr}_{₁} has an input comprising a signature of trusted authority 450 and an output locked to a public key of party 456 (e.g., an authoriser of a product/service, such as a doctor); tx_{Pt}_{₁} has an input comprising a signature of trusted authority 450 and an output locked to a public key of party 454 (e.g., a consumer of a product/service, such as a medical patient); tx_{Ph}_{₁} has an input comprising a signature of trusted authority 450 and an output locked to a public key of party 452 (e.g., a user of a product/service, such as a pharmacy).

FIG. 4 also shows an authorisation EPT at txₚᵣₛ_{₁}. In the specific example of FIG. 4, the authorisation event is a prescription issuing event between authoriser 456 (e.g., a doctor) and consumer 454 (e.g., a patient). The authorisation issuing event carries the authorisation details (e.g., prescription details) and is signed by the authoriser 456. The authorisation issuing event has an output that can be spent by consumer 454. In FIG. 4, txₚᵣₛ_{₁} has two inputs (in₁=tx_{Dr}_{₁}|0, in₂=tx_{Pt}_{₁}|0) and two spendable outputs (out₁=Address_{Dr}_{₁}, out₂=Address_{Pt}_{₁}) spendable only by authoriser 456 and consumer 454 respectively. In this event, the authoriser's signature is required. The consumer 454's signature is optional, yet it allows tracking the consumer's interactions on the transaction graph easier. An action may be performed, and in response to performing the action a signature of the party performing the action may be provided to unlock the output of the a previous blockchain transaction. The output of the previous blockchain transaction may then be used as an input to a blockchain transaction comprising information describing the action.

FIG. 4 also shows a collection EPT in tx_{col}_{₁}. EPT tx_{col}_{₁} contains signatures of product/service provider 452 (e.g., a pharmacy) and product/service consumer 454 (e.g., a patient), and details about the collected product/service (e.g., a prescription). The transaction has two inputs (in₁=tx_{Prs}_{₁}|1, in₂=tx_{Pharma}_{₁}|0) and two spendable outputs (out₁=Address_{Pt}_{₁}, out₂=Address_{Pharma}_{₁}).

In some examples, collection data (e.g., prescription details) can be hidden using OP_RETURN. Prescription details can be encrypted and inserted following an OP_RETURN. Either in a separate un-spendable output e.g.:
OP_FALSE OP_RETURN<cipher>,
or in the spendable output of the consumer 454 e.g.:
P2PKH<patient_Address>OP_RETURN<cipher>,
where cipher=Enc (key, m=<prescription>)

Encryption can be through the use of symmetric key based scheme (e.g., the Advanced Encryption Standard (AES)), and the shared encryption key can be determined from a Diffie Hellman Key exchange between consumer 454, and authoriser 456 or provider 452. The cipher can be decrypted by consumer 454, authoriser 456 and provider 452. They can do this using a key derived from their identity public key.

In some examples, the encryption key can be given by K_{symmetric}=Hash((sₚₐₜᵢₑₙₜK_{dr})|nonce)=Hash((s_{dr}Kₚₐₜᵢₑₙₜ)|nonce), where sₚₐₜᵢₑₙₜ, s_{dr} are the consumer 454's and authoriser 452's secret keys (scalar values), and Kₚₐₜᵢₑₙₜ, K_{dr} are their corresponding public keys (Elliptic curve points). The nonce is a value agreed by the patient and the doctor to ensure different encryption key is used in every transaction.

The public key pair used (s, K) can be selected to be the same key used to sign their transaction inputs.

It is possible that the encryption key may also have to be shared with a trusted authority 450 for the purpose of allowing access to the provider 452 and/or at least one auditor. In this case, we can optionally use three-party key agreement protocol DHP. For example the symmetric key can be given by

$${K}_{symmetric} = {s}_{dr} {s}_{patient} {s}_{TA} G = {s}_{dr} {s}_{patient} {K}_{TA} = {s}_{patient} {s}_{TA} {K}_{dr} = {s}_{dr} {s}_{TA} {K}_{patient}$$

In other examples, the collection details (e.g., prescription details) are cryptographically hashed and the hash value is inserted in the transaction, while the actual details are kept off-chain in a secure database.

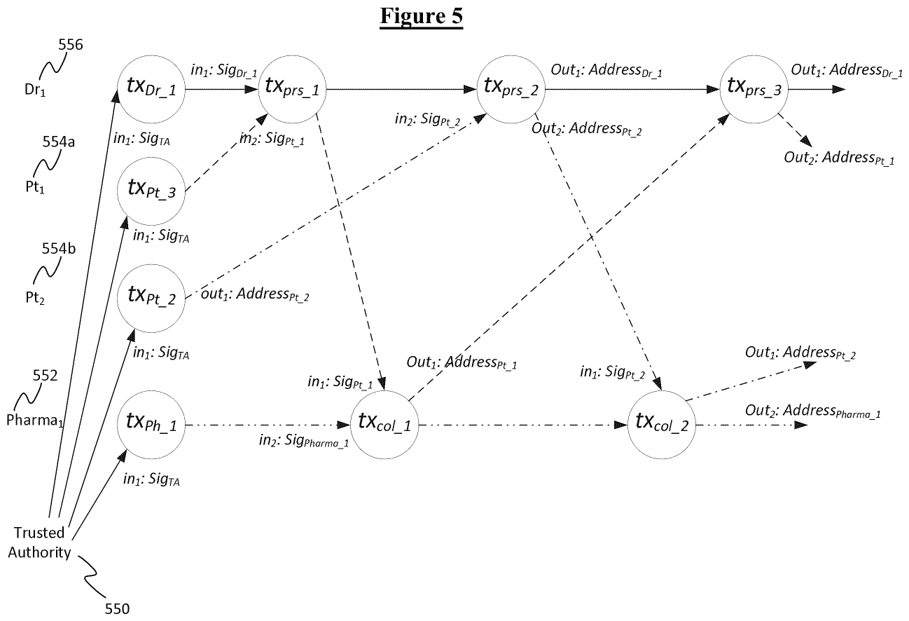

FIG. 5 shows another example comprising EPTs. FIG. 5 refers to a specific medical context example, with a doctor 556 (an authoriser of a product/service), two patients 554a and 554b (consumers of a product/service) a pharmacy 552 (a provider of a product/service) and a trusted authority 550. It will be understood however that the features of FIG. 5 may be generalised to any kind of EPTs that have a parent-child relationship. In particular, it will be understood that the features of FIG. 5 may be generalised to examples where a product/service is authorised, collected/used and provided.

When doctor 556 issues a new prescription to a patient 554a or 554b, she can trace all the patient's history (prescriptions issued and collected) by tracing the transactions graph for the patient.

Similarly, auditors checking the doctor 556's activity can trace the doctor 556's transactions graph and ensure that all prescriptions are issued to authenticated and allowed patients.

Auditors checking the pharmacist 552's activity can trace the pharmacist 552's transactions graph, and ensure all collected prescription were signed by authorised doctors (e.g., doctor 556) and given to authenticated patients (e.g., patient 554a and/or patient 554b).

In FIG. 5, it is possible to trace each actor's related events by following a corresponding transaction graph. Some event involve more than one actor and therefore there are transactions common in each actor's graph. FIG. 5 shows the following four transaction graphs:
Doctor 556's transaction graph is Tx_{Dr}_{₁}, Txₚᵣₛ_{₁}, Txₚᵣₛ_{₂}, Txₚᵣₛ_{₃} (connected by solid arrows).
First patient 554a's graph is Tx_{Pt}_{₁}, Txₚᵣₛ_{₁}, Tx_{col}_{₁}, Txₚᵣₛ_{₃} (connected by dash-dash arrows).
Second patient 554b's graph is Tx_{Pt}_{₂}, Txₚᵣₛ_{₂}, Tx_{col}_{₂} (connected by dash-dot-dash arrows)
Pharmacist 552's graph is Tx_{Ph}_{₁}, Tx_{col}_{₁}, Tx_{col}_{₂} (connected by dash-dot-dot-dash arrows)

When pharmacist 552 checks the doctor 556's authorisation and authentication, it is useful to do so without having to trace the doctor 556's graph transaction back to tx_{Dr}_{₁}. By linking the output of tx_{Dr}_{₁}, which is spendable by the doctor 556's private key, with the transaction carrying prescription data by referencing tx_{Dr}_{₁} in txₚᵣₛ_{ᵢ}. (i.e, by having a rule that tx_{Prs}_{ᵢ} includes the transaction identifier (txid) of tx_{Dr}_{₁}, pharmacist 552 (or another verifier) can get tx_{Dr}_{₁} and check that it is issued by trusted authority 550, attesting to the doctor 556's identity and check that the doctor 556's public key P_{Dr}_{₁} signed in tx_{Dr}_{₁} is related to the doctor 556's signature used in txₚᵣₛ_{ᵢ}. In other words, the transaction (tx_{Prs}_{ᵢ}) should be spending an output that requires the doctor 556's signature to allow this example method of checking the doctor 556's authorisation and authentication.

For example, if the doctor 556's certified key pair of tx_{Dr}_{₁} is P_{Dr}_{₁}, s_{Dr}_{₁}, then the doctor 556's private key to sign txₚᵣₛ_{ᵢ} can be given by s_{Dr}_{₁} or a function of s_{Dr}_{₁} such as s_{Dr}_{₁}+Hash(nonce_{sig}), where nonce_{sig} is made known to pharmacist 552. This nonce can be sent securely either off-chain by the doctor 556, shared with a trusted third party, or encrypted as a meta data inside the transaction itself. Pharmacist 552 checks that the public key used in the unlocking script in txₚᵣₛ_{ᵢ} is equal to P_{Dr}_{₁}+Hash(nonce_{sig}). G, and is satisfied that that is signed by doctor 556 identified in tx_{Dr}_{₁}. The same method should be applied to authenticate other signing actors. In some examples, the verification procedure is carried by a service provider.

A reused nonce affects the privacy of the signer as it enables observers to link the signer's different messages. It may also leak information about the signer's private key. A possible mitigation is to use unspent outputs as nonce, where its spend-status (spent/unspent) indicates validity, i.e. nonce=txid|index=outpoint. In such an example, doctor 556 could use a key given by s_{Dr}_{ᵢ}+Hash(outpointₓ), for example.

TABLE 1
Transaction creating a spendable output to be used as a
nonce in the transaction signing and is spent after usage
Tx_{nonce}

| | | | |
|---|---|---|---|
| Version | 1 | Locktime | 0 |
| In-count | — | Out-count | — |

| Input list | | | | |
|---|---|---|---|---|
| | Unlocking | Sequence | Output list | |
| Outpoint | script | Number | Value | Locking script |
| Any | | | Min | Address owned by |
| | | | | the signer |

Once outpoint, is used as nonce it should be spent, which could be done in the same (or different) transaction that carries the signature. To illustrate, the signer makes a transaction tx_{nonce}, creating an unspent output outpoint_{nonce}. The signer then makes the signature-carrying transaction tx_{sig} that spends outpoint_{nonce} in one input and the tx_{sig} includes a signature with private key s_{Dr}+Hash(outpoint_{nonce}), which can be in another input unlocking script or in OP_RETURN. The latter is shown in the following tables.

TABLE 2
Transaction that carries a signed message and
spends the output used as a nonce in the same time.
Tx_{signature}

| | | | |
|---|---|---|---|
| Version | 1 | Locktime | 0 |
| In-count | — | Out-count | — |

| Input list | | | | |
|---|---|---|---|---|
| | Unlocking | Sequence | Output list | |
| Outpoint | script | Number | Value | Locking script |
| TxId_{nonce}\|\|0 | | | 0 | OP_FALSE OP_RETURN < |
| | | | | data\|\|signature with key s_{Dr} + |
| | | | | Hash(TxId_{nonce}\|\|0)> |

### Immunity Against Replay Attacks by Ensuring Uniqueness of Signed Message

It should be noted that in some examples the implicit characteristic of a block transaction (spending an output only once) means that replay attacks are computationally infeasible, because every signed message should contain a string (the unspent output) which can be used only once. Messages signed using the signature in the unlocking script are therefore immune to replay attacks. In the case where the signature is embedded as a separate data string in the transaction (i.e. following an OP_RETURN), the signed message should include the outpoints that are spent in the transaction as a form of nonce. i.e. the signed message contains outpoint.

### 3.2 “Second Layer Solution”

One unwanted aspect of the solution discussed in Section 3.1 is that it is possible to trace all actor activities and interactions by following their transaction graph. For example, in the example of FIG. 5, it is relatively easy to trace the number of prescriptions given by a doctor since registration, the number of doctor's patients and the number of prescriptions issued for each patient. When the solution is applied more generally, it is relatively easy to tell how many authorisations of a product/service an authoriser has made, how many consumers and authoriser is responsible for, and how many products/services have been issued for each consumer by a provider. In addition to the privacy aspect, each actor has to have a wallet to sign and receive transactions, which incur extra layer of complexity.

In an example ‘second layer solution’, the actors' signatures are inserted as data included within the transaction, and the locking and unlocking scripts of the transactions (presc, collect, etc) are generated by a service provider (e.g. Kensei), independent of the actor.

We assume that the service provider has a cache of unspent transaction outputs (linking transaction outputs) ready to be used in creating events-carrying transactions.

It should be noted that most of these linking transaction outputs are expected to be easily linked to the service provider, which may pose a privacy risk in some examples. However, privacy can be maintained through scaling. Increasing number of events and clients make it harder for an outsider to associate which transactions carry which events for which client.

### Linking Actor's Events

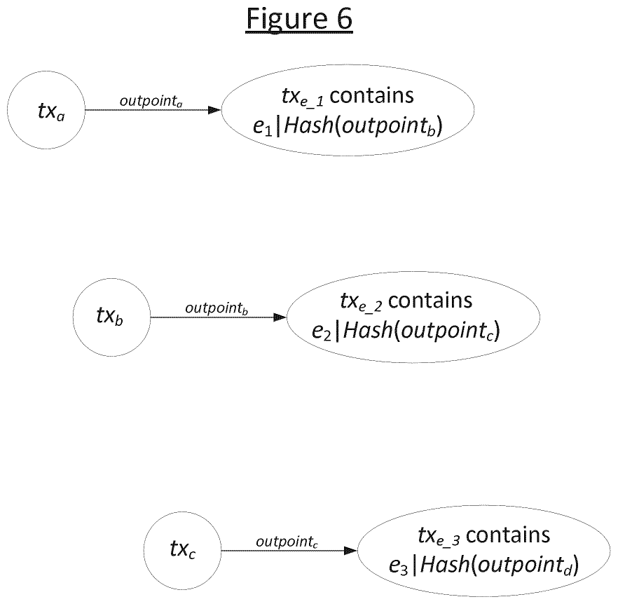

Suppose an actor (can be an authoriser of a product/service, a consumer of a product/service, a provider of a product/service or in a medical context could be a doctor, patient or pharmacist) is involved in the following sequence of events e₁, e₂, e₃, which are published in txₑ_{₁}, txₑ_{₂}, txₑ_{₃}, respectively. Note that these transactions may be in the same transaction chain but not immediately have to be spending one another (do not have a child-parent relationship). As shown in FIG. 6, a connection between these events can be established privately on-chain by including in txₑ_{₁} (containing information of a current event e₁) the hash of the outpoint that is going to be spent in the transaction txₑ_{₂} (containing the next event e₂). Thus txₑ_{₁}, for example contains (e₁, Hash(outpoint))), and txₑ_{₂} spends outpoint; and contains the next event (e₂). A connection between these events can be established privately on-chain by including in txₑ_{₂} (containing information of a current event e₂) the hash of the outpoint that is going to be spent in the transaction txₑ_{₂} (containing the next event e₃). Thus txₑ_{₂}, for example contains (e₂, Hash(outpoint)), and txₑ_{₂} spends outpoint, and contains the next event (e₃).

As shown in FIG. 6, when a first party performs a first action e₁, a first signature of the first party can be included as data in a first event transaction txₑ_{₁}. The first event transaction may include a hash of a first outpoint (outpoint_{b}) of a first linking transaction tx_{b}. When the first party performs a second action, a second signature of the first party is included as data in a second event transaction txₑ_{₂}, where the second event transaction txₑ_{₂} includes the first outpoint (outpoint_{b}) as an input.

The second event transaction txₑ_{₂} may comprise a second outpoint (outpoint_{c}) of a second linking transaction. In response to the first party performing a third action, a third signature of the first party may be included as data in a third event transaction, where the third event transaction comprises the second outpoint (outpoint_{c}) as an input. The first outpoint (outpoint_{b}) and second outpoint (outpoint_{c}) may be generated by the service provider.

An off-chain solution would be to store the index of txₑ_{₁}, txₑ_{₂}, txₑ_{₃}, against the actor's record e₁, e₂, e₃.

If the service provider mistakenly spent an outpoint outpoint_{b} in an unrelated event [symbol] +e₂, there may be a mechanism to detect and correct errors. Some example mechanisms are:
1. Application level-solutions embedded in the events eᵢ:
   a. Use of error correction and detection codes to detect and correct errors
   b. Use of authentication techniques
2. Service provider level
   a. Off-chain records should have flags to indicate if an error happened.
   b. Include the hash of the address being spent rather than (or in addition to) the outpoint itself, i.e. Hash(P2PKH<P_{b}>) for example.
   c. In case of mapping a wrong event êₓ in a wrong transaction, the service provider publishes a correction transaction which would include signatures linked to signatures used in the past correct transactions to prove authenticity of the correcting transaction. For e.g. if s₁, s₂ are secret keys used in signing message-publishing transactions t₁, t₂, by the service provider, then the service provider can prove oneself as the signer of these transactions by creating two signatures using s₁+nonce₁ and s₂+nonce₂, in the correcting-transaction and send (nonce₁, nonce₂) to the verifier.

In some examples, fabricated events are inserted in transactions in a manner that does not allow attackers to determine the real information among the fake spurious data. These examples employ misdirection to confuse potential attackers.

### 3.3 Proving Existence of Events

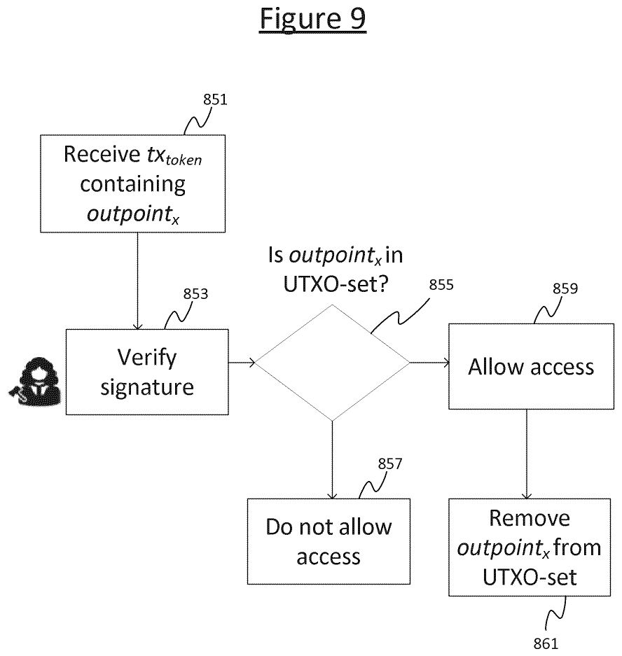

Two methods of mapping events from event streams to blockchain transactions are discussed above.
1. Event-publishing-Transactions (EPT) are linked such that one is spending the other. An example of this was shown in 3.1 (first layer solution). In this case, it is possible to trace events by tracing the transaction graph, since all EPT of an event stream are linked by a child-parent relationship. In FIG. 9, event e_{A}_{ᵢ} is published in

$${tx}_{{e}_{{A}_{i}}} .$$

It is possible to trace all events by following the transaction graph.
2. EPTs of an event stream are not linked by child-parent relationship as discussed in 3.2. As has been shown in FIG. 6. This design choice provides privacy since an external observer is not able to easily trace EPTs of an event stream.

When providing the proof of existence for an event, we provide to a second party the Merkle proof of the EPT of that event, as well as the raw-transaction itself; this is to show the event data (or the event hash if the hash of the event is what is published). If the EPTs of an event stream have a child-parent relationship, we can replace the Merkle proofs for all transaction by providing the Merkle proof of the most recent child transaction only. This can be a welcome optimisation when the number of events is large. To illustrate (See Error! Reference source not found.), if we want to prove the existence of events e₁ to e₄, the following may be provided:
1. Merkle proof of

$${tx}_{{e}_{{A}_{4}}}$$

2. Raw transaction format of

$${tx}_{{e}_{{A}_{1}}}$$

to

$${tx}_{{e}_{{A}_{4}}}$$

However in the case where the EPTs are not linked with a child-parent relationship, the following may need to be provided:
1. Merkle proof of each EPT
2. Raw transaction of each EPT

It is possible to reduce the size of the proof by creating a transaction that spends an output from all EPTs. We call this transaction a child-of-all transaction (tx_{coa}) or event summary transaction-see FIG. 8. In this case, all events can be proved by providing:
1. Merkle proof of the tx_{coa}
2. Raw transaction format of the tx_{coa}
3. Raw transaction of each EPT

This reduces the size of the proof. This solution links all EPT transactions on the blockchain. To counter privacy concerns, misdirection technique techniques can also be employed (for example fake EPTs can be used to provide tx_{coa} along with real EPTs).

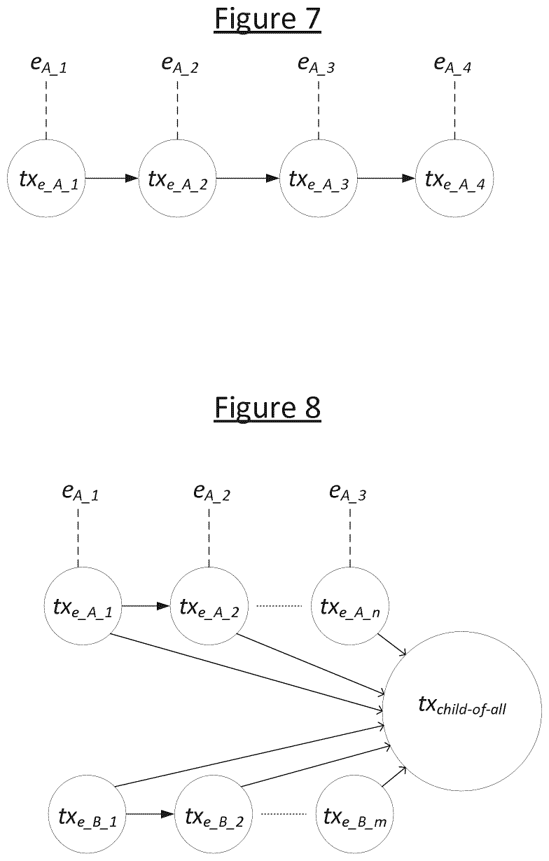

In some examples the tx_{coa} can spend outputs from all EPTs from all different streams. Using a service provider (such as Kensei) that publishes events from different streams in blockchain transaction, it is possible to use this method to provide a degree of privacy. In FIG. 7, we have two event streams A and B. There is no child-parent relationship between any two EPT transactions. We create a tx_{coa} that spends an output of each EPT. Event stream A comprises e_{A}_{₁}, e_{A}_{₂}, . . . , e_{A}_{ₙ}. Event stream B comprises e_{B}_{₁}, e_{B}_{₂}, . . . , e_{B}_{ₘ}.

One of the advantages of the tx_{coa} is that it is common to all event streams whose EPTs have an outpoints spent in it. i.e. tx_{coa} can be easily shared between different customers of the service provider. This becomes like block header chain data, that must be provided along with the Merkle proof when proving the existence of any transaction.

However, the size of the tx_{coa} can be very large especially when we want to preserve privacy by combining outpoints from EPT from different streams.

Moreover, we need to have a spendable outpoint in each EPT. It is possible to insert the event data in a spendable output, without having to create a new data-specific output in each EPT. One way to reduce the size of the unlocking script in tx_{coa} is to use a Hash function instead of OP_CHECKSIG. The locking script can be in the form of:
OP_HASH160<RIPEMD160(SHA256 (v))>

To which the unlocking script can be in the form of:
<v>

The minimum length of v should be 128-bits (16 bytes). This reduces the size of the unlocking script from 106.5-byte (used in P2PKH) to only 16 bytes. The average size of P2PKH inputs is about 147.5 bytes: 40 bytes for referencing UTXO and the sequence number, a one-byte variable-length integer to encode the unlocking script's size, and the 106.5 byte unlocking script. The average size now becomes 57 bytes. This also reduces the size of the locking script (in an EPT) from 25-byte (locking script in P2PKH) to about 20 bytes.

In addition to replacing OP_CHECKSIG with OP_HASH160, the value (coins) of the outpoint (from each EPT) should be as minimum as possible (say one Satoshi). This is because removing OP_CHECKSIG allows any entity to spend that output in a different transaction. This attack can only be carried in the time before the tx_{coa} is confirmed. To mitigate this risk we make the value of the outpoint so low such that it is not worth for an attacker to carry such an attack. The size of tx_{coa} is large enough that it requires spending a P2PKH input to fund the transaction and pay the miner's fee. See Error! Reference source not found.

The unlocking scripts in the tx_{coa} in Error! Reference source not found. from the EPT is given by v_{A}_{₁}, v_{A}_{₂}, . . . , v_{B}_{ₙ}. It is possible to have a single vᵢ value (per stream or across all streams) to allow compact off-chain storage and transmission of the tx_{coa}. Alternatively, it is possible to have a deterministic relationship between different v_{A}_{ᵢ}, such that compression is still possible. For example v_{A}_{ᵢ}=Hash(v_{A}_{_{i−1}}|secret_{A}), or v_{A}_{ᵢ}=Enc(counterᵢ|Key_{A}).

TABLE 3
Child-of-all transaction
Tx_{child-of-all}

| Version | 1 | | Locktime | 0 |
|---|---|---|---|---|
| In-count | — | | Out- | — |
| | | | count | |
| Input list | | | Output list | |
| | | Sequence | | |
| Outpoint | Unlocking script | Number | Value | Locking script |
| ${Tx}_{{e}_{{A}_{1}}} \vert \vert 0$ | v_{A}_{₁} | | | |
| ... | v_{A}_{₂} | | | |
| ${Tx}_{{e}_{{A}_{n}}} \vert \vert 0$ | | | | |
| ... | ... | | | |
| ${Tx}_{{e}_{{B}_{m}}} \vert \vert 0$ | v_{B}_{ₘ} | | | |
| Tx_{fee}\|\|i | < sig >< Pub> | | | |

In some examples, a child-of-all transaction at time Tᵢ,

$${T}_{i} , {tx}_{{coa}_{{T}_{i}}} ,$$

can be created, whose Merkle proof proves the existence of all EPT which had an output spent in it. The

$${tx}_{{coa}_{{T}_{i}}}$$

and

$${tx}_{{coa}_{{T}_{i - 1}}}$$

may have a child-parent relationship. We constantly replace the Merkle proof by that of the most recent

$${tx}_{{coa}_{{T}_{i}}} .$$

Thus we can prove the existence of any EPT as long as we have the raw transaction of all

$${tx}_{{coa}_{Ti}}$$

up to the most recent, along with its Merkle proof.

### 3.4 Access Control Using Transactions

In this section we assume we have a database that holds records (e.g., patients' records).

In some examples, there are 3 different actors: an oracle, the patient and the doctor (or pharmacist). The oracle is responsible for allowing and preventing access to the database. The patient holds the right to grant access rights to his records. The oracle is responsible to enforce access controls. The doctor and the pharmacist want to access the data.

An example use case if for a patient to grant access rights to a doctor (or pharmacist) to read or edit the patient's records.

The database contains the patient's records and the patient's public key P_{Pt}. The patient may prove ownership of the public key by presenting a signature corresponding to that public key to the oracle. The doctor (or pharmacist) is granted access to the patient's record by the oracle only when the patient's signature provided corresponds to the public key. The patient's public key can be used to identify the patient's record. It is also possible to use Hash(P_{Pt}) instead of P_{Pt}

The patient wants to send an access token to the doctor. The oracle grants access when presented with valid tokens. The access token may expire after usage.

The transaction signed by the patient may comprise the token. An outpoint of the transaction is used to represent the validity of the token. If the outpoint is unspent then the token is valid, otherwise it is invalid.

### Outpoint as a Database Access Control Token

The signed message contains the hash of unspent output m=Hash(outpoint,), which (the outpoint) will be spent by the agent after it grants access. Once outpoint, is spent, the token is deemed expired. See FIG. 9. Here the patient signs the message m and passes it to the doctor (or the pharmacist) to allow access. At 851, the doctor/pharmacist sends the signed message m to the oracle (access agent) along with the access request. At 853, the oracle checks the signature of the patient. At 855, the oracle checks that outpoint, is in the UTXO-set, and if it is the agent grants access to the doctor/pharmacist at 859, and spends outpoint, to remove it from the UTXO-set at 861. If outpoint, is not in the UTXO-set, the oracle denies access to the doctor/pharmacist at 857.

There are also the following exemplary options:
a. The output outpoint, is spendable by the agent or the patient (1-out-2 multisig). This allows any of the two to render the token invalid, or revoke access.
b. The signed message is the concatenation of the spendable outputs m=hash (outpointₓ|outpoint_{y}), where one is spendable by the patient and the other is spendable by the agent. Here the access is granted using one of these rules.
   i. Access is granted as long as both outputs are unspent, and denied otherwise
   ii. Access is denied only when both outputs are spent.

### Outpoint as an Identity Hiding Token

The oracle may announce securely an outpoint_{T}=txid|index spendable by the agent periodically. When communicating with the agent. The patient signs a message using the s_{Pt}+Hash(outpoint_{T}). The agent checks if the corresponding public key is P_{Pt}+Hash(out_{T})·G. and that outpoint is in the UTXO-set. At the next time interval, the oracle spends out_{T}, and selects a new unspent output as the next token out_{T+1}.

### 4. Further Remarks

Other variants or use cases of the disclosed techniques may become apparent to the person skilled in the art once given the disclosure herein. The scope of the disclosure is not limited by the described embodiments but only by the accompanying claims.

For instance, some embodiments above have been described in terms of a bitcoin network 106, bitcoin blockchain 150 and bitcoin nodes 104. However it will be appreciated that the bitcoin blockchain is one particular example of a blockchain 150 and the above description may apply generally to any blockchain. That is, the present invention is in by no way limited to the bitcoin blockchain. More generally, any reference above to bitcoin network 106, bitcoin blockchain 150 and bitcoin nodes 104 may be replaced with reference to a blockchain network 106, blockchain 150 and blockchain node 104 respectively. The blockchain, blockchain network and/or blockchain nodes may share some or all of the described properties of the bitcoin blockchain 150, bitcoin network 106 and bitcoin nodes 104 as described above.

In preferred embodiments of the invention, the blockchain network 106 is the bitcoin network and bitcoin nodes 104 perform at least all of the described functions of creating, publishing, propagating and storing blocks 151 of the blockchain 150. It is not excluded that there may be other network entities (or network elements) that only perform one or some but not all of these functions. That is, a network entity may perform the function of propagating and/or storing blocks without creating and publishing blocks (recall that these entities are not considered nodes of the preferred bitcoin network 106).

In other embodiments of the invention, the blockchain network 106 may not be the bitcoin network. In these embodiments, it is not excluded that a node may perform at least one or some but not all of the functions of creating, publishing, propagating and storing blocks 151 of the blockchain 150. For instance, on those other blockchain networks a “node” may be used to refer to a network entity that is configured to create and publish blocks 151 but not store and/or propagate those blocks 151 to other nodes.

Even more generally, any reference to the term “bitcoin node” 104 above may be replaced with the term “network entity” or “network element”, wherein such an entity/element is configured to perform some or all of the roles of creating, publishing, propagating and storing blocks. The functions of such a network entity/element may be implemented in hardware in the same way described above with reference to a blockchain node 104.

Some embodiments have been described in terms of the blockchain network implementing a proof-of-work consensus mechanism to secure the underlying blockchain. However proof-of-work is just one type of consensus mechanism and in general embodiments may use any type of suitable consensus mechanism such as, for example, proof-of-stake, delegated proof-of-stake, proof-of-capacity, or proof-of-elapsed time. As a particular example, proof-of-stake uses a randomized process to determine which blockchain node 104 is given the opportunity to produce the next block 151. The chosen node is often referred to as a validator. Blockchain nodes can lock up their tokens for a certain time in order to have the chance of becoming a validator. Generally, the node who locks the biggest stake for the longest period of time has the best chance of becoming the next validator.

It will be appreciated that the above embodiments have been described by way of example only. More generally there may be provided a method, apparatus or program in accordance with any one or more of the following Statements.

Statement 1: A method performed by a first party, the method comprising: receiving, from a second party, information of a first blockchain transaction, the first blockchain transaction comprising an input and an output, wherein the input of the first blockchain transaction comprises a signature of the second party and wherein the output of the first blockchain transaction is locked to a public key of the first party; performing an action comprising at least one of: receiving authorisation of access to a product or service; issuing authorisation of access to a product or service; providing a product or service; receiving a product or service; and wherein the method comprises: in response to performing the action, providing, as part of a first input of a second blockchain transaction, a signature of the first party to unlock the output of the first blockchain transaction.

Statement 2: A method according to Statement 1, wherein the second party comprises a trusted authority.

Statement 3. A method according to Statement 1 or Statement 2, wherein a second input of the second blockchain transaction comprises a signature of a third party.

Statement 4. A method according to Statement 3, wherein the second blockchain transaction comprises a first output locked to a public key of the third party.

Statement 5. A method according to Statement 3 or Statement 4, wherein the second blockchain transaction comprises a second output locked to a second public key of the first party.

Statement 6. A method according to any of Statements 3 to 5, wherein: the first party comprises an authoriser of access to a product or service and the third party comprises a user of the product or service; or the first party comprises the user of the product or service and the third party comprises authoriser of access to the product or service.

Statement 7. A method according to any of Statements 3 to 5, wherein: the first party comprises a provider of a product or service and the third party comprises a user of the product or service; or the first party comprises the user of the product or service and the third party comprises the provider of the product or service.

Statement 8. A method according to Statement 4 or Statement 5, comprising: signing an output of the second blockchain transaction and providing the signed first output of the second blockchain transaction as an input of a third blockchain transaction, wherein the third blockchain transaction comprises: a further input signed by a fourth party; a first output requiring a signature of the first party in order to spend the first output of the third blockchain transaction; a second output requiring a signature of the fourth party in order to spend the second output of the third blockchain transaction.

Statement 9. A method according to Statement 8, wherein: the first party comprises a user of a product or service; the third party comprises an authoriser of access to the product or service; the fourth party comprises a provider of the product or service.

Statement 10. A method according to any preceding Statement, wherein the method comprises at least one of: submitting the second blockchain transaction to one or more nodes of a blockchain; sending the second blockchain transaction to a fifth party to send to one or more nodes of a blockchain.

Statement 11. A method according to any preceding Statement, wherein the first blockchain transaction comprises data describing the action or an encrypted version thereof.

Statement 12. A method according to Statement 11 when dependent on Statement 3, wherein the data is encrypted using an encryption key derivable by the first party and the third party.

Statement 13. A method according to any preceding Statement, wherein the second blockchain transaction and/or a fourth blockchain transaction comprises a transaction identifier of the first blockchain transaction.

Statement 14. A method according to any preceding Statement, wherein the method comprises: determining a private key of the first party for a fifth blockchain transaction as a function of a private key of the first party for the first blockchain transaction and a hash of a nonce value; sending the nonce value to the second party.

Statement 15. A method according to Statement 14, wherein an output of the fifth blockchain transaction is locked to a public key corresponding to the private key of the first party for the fifth blockchain transaction.

Statement 16. A method according to Statement 14 or Statement 15, the method comprising: generating a nonce transaction, the nonce transaction comprising an output, wherein the nonce value is based on a transaction identifier of the nonce transaction and an index of the output.

Statement 17. Computer equipment comprising: memory comprising one or more memory units; and processing apparatus comprising one or more processing units, wherein the memory stores code arranged to run on the processing apparatus, the code being configured so as when on the processing apparatus to perform the method of any of Statements 1 to 16.

Statement 18. A computer program embodied on computer-readable storage and configured as, when run on one or more processors, to perform the method of any of Statements 1 to 16.

According to another aspect disclosed herein, there may be provided a method comprising the actions of the first party. According to another aspect disclosed herein, there may be provided a system comprising the computer equipment of the first party.

According to another aspect disclosed herein, there may be provided a method comprising the actions of the second party. According to another aspect disclosed herein, there may be provided a system comprising the computer equipment of the second party.

According to another aspect disclosed herein, there may be provided a method comprising the actions of the third party. According to another aspect disclosed herein, there may be provided a system comprising the computer equipment of the third party.

1. A method performed by a first party, the method comprising:
receiving, from a second party, information of a first blockchain transaction, the first blockchain transaction comprising an input and an output, wherein the input of the first blockchain transaction comprises a signature of the second party and wherein the output of the first blockchain transaction is locked to a public key of the first party;
performing an action comprising at least one of: receiving authorisation of access to a product or service; issuing authorisation of access to a product or service; providing a product or service; receiving a product or service; and wherein the method comprises:
in response to performing the action, providing, as part of a first input of a second blockchain transaction, a signature of the first party to unlock the output of the first blockchain transaction, wherein a second input of the second blockchain transaction comprises a signature of a third party and the second blockchain transaction comprises a second output locked to a second public key of the first party.

2. The method of claim 1, wherein the second party comprises a trusted authority.

3. (canceled)

4. The method of claim 1, wherein the second blockchain transaction comprises a first output locked to a public key of the third party.

5. (canceled)

6. The method of claim 1, wherein:
the first party comprises an authoriser of access to a product or service and the third party comprises a user of the product or service; or
the first party comprises the user of the product or service and the third party comprises authoriser of access to the product or service.

7. The method of claim 1, wherein:
the first party comprises a provider of a product or service and the third party comprises a user of the product or service; or
the first party comprises the user of the product or service and the third party comprises the provider of the product or service.

8. The method of claim 1, comprising:
signing an output of the second blockchain transaction and providing the signed output of the second blockchain transaction as an input of a third blockchain transaction, wherein the third blockchain transaction comprises:
a further input signed by a fourth party;
a first output requiring a signature of the first party in order to spend the first output of the third blockchain transaction; and
a second output requiring a signature of the fourth party in order to spend the second output of the third blockchain transaction.

9. The method of claim 8, wherein:
the first party comprises a user of a product or service;
the third party comprises an authoriser of access to the product or service; and
the fourth party comprises a provider of the product or service.

10. The method of claim 1, wherein the method comprises at least one of:
submitting the second blockchain transaction to one or more nodes of a blockchain; and
sending the second blockchain transaction to a fifth party to send to one or more nodes of a blockchain.

11. The method of claim 1, wherein the first blockchain transaction comprises data describing the action or an encrypted version thereof.

12. The method of claim 11, wherein the data is encrypted using an encryption key derivable by the first party and the third party.

13. The method of claim 1, wherein the second blockchain transaction and/or a fourth blockchain transaction comprises a transaction identifier of the first blockchain transaction.

14. The method of claim 1, wherein the method comprises:
determining a private key of the first party for a fifth blockchain transaction as a function of a private key of the first party for the first blockchain transaction and a hash of a nonce value; and
sending the nonce value to the second party.

15. The method of claim 14, wherein an output of the fifth blockchain transaction is locked to a public key corresponding to the private key of the first party for the fifth blockchain transaction.

16. The method of claim 14, the method comprising:
generating a nonce transaction, the nonce transaction comprising an output, wherein the nonce value is based on a transaction identifier of the nonce transaction and an index of the output.

17. Computer equipment, comprising:
memory comprising one or more memory units; and
processing apparatus comprising one or more processing units, wherein the memory stores code arranged to run on the processing apparatus, the code being configured so as when run on the processing apparatus, the processing apparatus performs a method performed by a first party, the method comprising:
receiving, from a second party, information of a first blockchain transaction, the first blockchain transaction comprising an input and an output, wherein the input of the first blockchain transaction comprises a signature of the second party and wherein the output of the first blockchain transaction is locked to a public key of the first party;
performing an action comprising at least one of: receiving authorisation of access to a product or service; issuing authorisation of access to a product or service; providing a product or service; receiving a product or service; and wherein the method comprises:
in response to performing the action, providing, as part of a first input of a second blockchain transaction, a signature of the first party to unlock the output of the first blockchain transaction, wherein a second input of the second blockchain transaction comprises a signature of a third party and the second blockchain transaction comprises a second output locked to a second public key of the first party.

18. A non-transitory computer readable medium, comprising a computer program configured so as, when run on one or more processors, the one or more processors perform a method performed by a first party, the method comprising:
receiving, from a second party, information of a first blockchain transaction, the first blockchain transaction comprising an input and an output, wherein the input of the first blockchain transaction comprises a signature of the second party and wherein the output of the first blockchain transaction is locked to a public key of the first party;
performing an action comprising at least one of: receiving authorisation of access to a product or service; issuing authorisation of access to a product or service; providing a product or service; receiving a product or service; and wherein the method comprises:
in response to performing the action, providing, as part of a first input of a second blockchain transaction, a signature of the first party to unlock the output of the first blockchain transaction, wherein a second input of the second blockchain transaction comprises a signature of a third party and the second blockchain transaction comprises a second output locked to a second public key of the first party.
